(19) United States
(12) Patent Application Publication
(10) Pub. No.: US 2019/0060189 A1
(43) Pub. Date: Feb. 28, 2019

(54) METHOD OF LUBRICATING AN ORAL APPLIANCE

(71) Applicant: Eversmile, Los Angeles, CA (US)

(72) Inventor: Michael J. Florman, Pacific Palisades, CA (US)

(21) Appl. No.: 16/175,761

(22) Filed: Oct. 30, 2018

Related U.S. Application Data
(63) Continuation-in-part of application No. 15/398,706, filed on Jan. 4, 2017, now abandoned.
Application No. 15/398,706 is a continuation of application No. 14/271,174, filed on May 6, 2014, now Pat. No. 9,561,161.
(60) Provisional application No. 61/820,393, filed on May 7, 2013.

(51) Int. Cl. A61K 8/22; A61Q 11/02; A61Q 11/00; A61K 8/04

(52) U.S. Cl. CPC A61K 8/22; A61Q 11/02; A61K 2800/87; A61K 2800/805; A61Q 11/00; A61K 8/046

(57) ABSTRACT

Described is a method for lubricating an oral appliance which reduces friction at points of contact in the mouth. A quantity of a formulation is introduced into the user's mouth with the formulation being directed onto the appliance. The formulation is applied in such a way that the formulation will reside between the appliance and the user's teeth. The formulation is aimed close to areas of contact between the user's teeth and the appliance. The formulation is kept in the mouth for a desired amount of time. The formulation comprises a lubricant composition.

### CROSS-REFERENCE TO RELATED APPLICATIONS

This is a Continuation-in-Part application of U.S. Non-Provisional patent application Ser. No. 15/398,706, filed in the United States on Jan. 4, 2017, entitled, “Method of Cleaning an Oral Appliance,” which is a Continuation Application of U.S. Non-Provisional patent application Ser. No. 14/271,174, filed in the United States on May 6, 2014, entitled, “Method of Cleaning an Oral Appliance,” which is a Non-Provisional patent application of U.S. Provisional Application No. 61/820,393, filed on May 7, 2013, entitled, “Method of Cleaning an Oral Appliance,” the entirety of which are hereby incorporated by reference.

### BACKGROUND OF THE INVENTION

### (1) Field of Invention

The present invention relates to a method for lubricating an oral appliance and, more particularly, to a method for lubricating an oral appliance by directing a formulation at friction points in the mouth.

### (2) Description of Related Art

Oral appliances, such as orthodontic retainers, dental aligners (such as Invisalign®, ClearCorrect™, and other brands), dentures, sleep apnea appliances, sports mouth guards, etc. that are worn for a length of time can often be uncomfortable and/or not function properly due to friction between the oral appliance and the mouth or teeth. Thus, a need exists for a lubricating formulation that can be used on the go, throughout the day if necessary, to enhance the fit or movements of the oral appliance and/or increase comfort in the wearer's mouth.

### SUMMARY OF THE INVENTION

The present invention relates to a method for lubricating an oral appliance and, more particularly, to a method for lubricating an oral appliance by directing a formulation at friction points in the mouth. The method comprises acts of introducing a quantity of a formulation into the user's mouth, the formulation being directed onto the appliance; applying the formulation in such a way that the formulation will reside between the appliance and at least one of the user's teeth, wherein the formulation is aimed close to areas of contact between at least one of the user's teeth and the appliance; and keeping the formulation in the mouth for a desired amount of time, wherein the formulation comprises a lubricant composition.

In another aspect, the formulation is applied via a syringe between the appliance and at least one of the user's teeth.

In another aspect, the formulation is applied via a syringe between the appliance and an orthodontic attachment affixed to at least one of the user's teeth.

In another aspect, the formulation is applied via a spray mechanism.

In another aspect, the formulation is applied via a foaming mechanism.

The present invention further relates to a method of lubricating an oral appliance while the appliance is in place in a user's mouth, comprising act of introducing a quantity of a formulation into the user's mouth in such a way that the formulation will reside between the appliance and at least one of the user's teeth; keeping the formulation in the mouth for a desired amount of time, wherein the formulation comprises a lubricant composition.

In another aspect, the formulation is introduced into the user's mouth in powder form, dissolved in the user's mouth, resulting in a dissolved formulation, and dispersed around the appliance.

In another aspect, a brush is used to spread the formulation onto the appliance.

The present invention further relates to a method of lubricating an oral appliance, comprising acts of applying a quantity of a formulation in or onto the appliance while it is removed from a mouth in particular on portions of the appliance that come into contact with at least one of a user's teeth when the appliance is in the user's mouth; inserting the appliance into the user's mouth; allowing the formulation to reside between the appliance and at least one of the user's teeth for a selected period of time, wherein the formulation comprises a lubricant composition.

### BRIEF DESCRIPTION OF THE DRAWINGS

The objects, features and advantages of the present invention will be apparent from the following detailed descriptions of the various aspects of the invention in conjunction with reference to the following drawings, where:

FIG. 1 is a flowchart illustrating three embodiments of a method of lubricating an oral appliance according to some embodiments of the present disclosure;

FIG. 2 is an illustration of the application of a foamed lubricant formulation to an oral appliance according to some embodiments of the present disclosure;

FIG. 3 is an illustration of the application of a lubricant formulation to an oral appliance outside of the mouth according to some embodiments of the present disclosure;

FIG. 4 is an illustration of the application of a spray lubricant formulation to an oral appliance inside the mouth according to some embodiments of the present disclosure;

FIG. 5 is an illustration of the application of a lubricant formulation to an oral appliance inside the mouth via syringe according to some embodiments of the present disclosure;

FIG. 6 is an illustration of the application of a lubricant formulation in powder form to an oral appliance inside the mouth according to some embodiments of the present disclosure;

FIG. 7 is an illustration of the application of a lubricant formulation to an oral appliance outside the mouth via syringe according to some embodiments of the present disclosure;

FIG. 8A is an illustration of a fixed oral appliance in the mouth according to some embodiments of the present disclosure; and

FIG. 8B is an illustration of aligner attachments and a removable aligner according to some embodiments of the present disclosure.

### DETAILED DESCRIPTION

The following description is presented to enable one of ordinary skill in the art to make and use the invention and to incorporate it in the context of particular applications. Various modifications, as well as a variety of uses, in different applications will be readily apparent to those skilled in the art, and the general principles defined herein may be applied to a wide range of embodiments. Thus, the present invention is not intended to be limited to the embodiments presented, but is to be accorded with the widest scope consistent with the principles and novel features disclosed herein.

In the following detailed description, numerous specific details are set forth in order to provide a more thorough understanding of the present invention. However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

Please note, if used, the labels left, right, front, back, top, bottom, forward, reverse, clockwise and counter-clockwise have been used for convenience purposes only and are not intended to imply any particular fixed direction. Instead, they are used to reflect relative locations and/or directions between various portions of an object. As such, as the present invention is changed, the above labels may change their orientation.

(1) Specific Details of the Invention

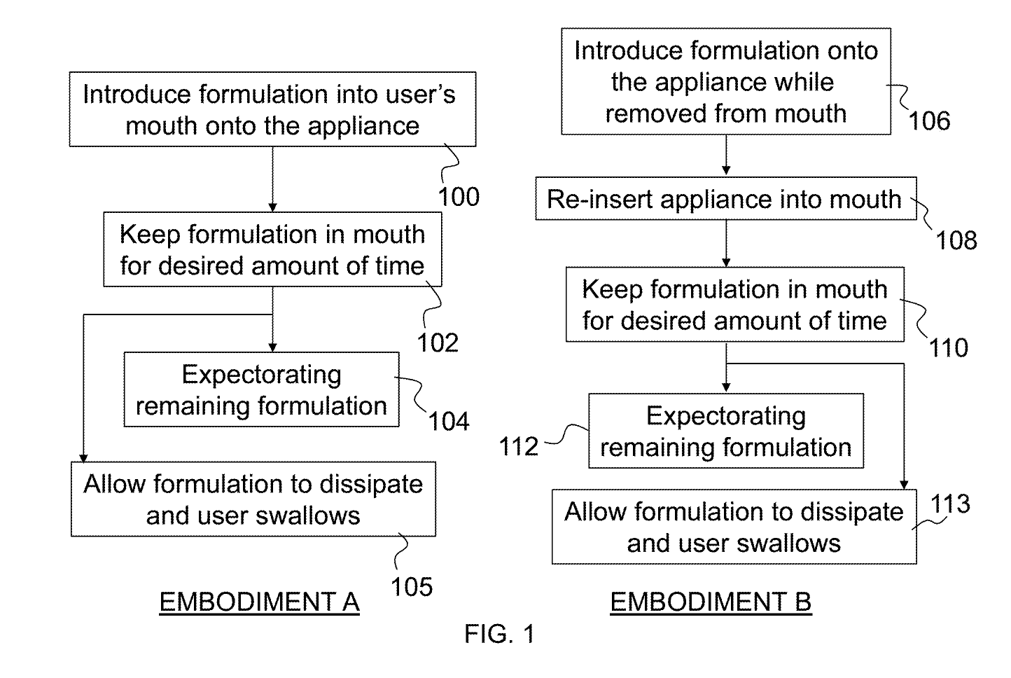

According to a first aspect of the invention, a method of lubricating an oral appliance is provided that allows a dental patient or other oral appliance wearer to lubricate the appliance either while the appliance is in place in the user's mouth or outside the mouth. FIG. 1 illustrates two embodiments of the lubricating method. In embodiment A, the oral appliance wearer applies the lubricant formulation directly to the appliance while the appliance is being worn (element 100); keeps the formulation in the mouth for a desired amount of time (element 102), perhaps swishing the lubricant formulation around in the mouth for a short period of time; and, if necessary, expectorates any excess lubricant formulation (element 104). Alternatively, the lubricant formulation can be allowed to dissipate within the user's mouth where it is eventually swallowed (element 105). In this embodiment, the lubricant formulation can be applied via a foaming mechanism where the formulation facilitates foaming, such as dispensed from a foam-dispensing bottle and applied to the appliance as a foam, applied via a syringe, sprayed, or dispensed in powder, gel, or liquid form.

In embodiment B, after removing the appliance from his or her mouth, the appliance wearer dispenses a quantity of lubricant formulation into or onto the appliance (element 106); reinserts the appliance back into his or her mouth (element 108); keeps the appliance and formulation into the mouth (element 110); and, if necessary, expectorates any excess lubricant formulation (element 112). Alternatively, the lubricant formulation can be allowed to dissipate within the user's mouth where it is eventually swallowed (element 113). As described below in greater detail, the lubricant formulation can be dispensed from a foam-dispensing bottle and applied to the appliance as a foam, applied via a syringe, or sprayed. Additionally, it is possible that after removing the appliance from his or her mouth, the appliance wearer applies the cleanser formulation to the appliance (element 106); places the appliance into a storage case and, after allowing the appliance to dwell for some time (e.g., 10 to 60 seconds or 5 to 30 minutes), retrieves the appliance from the storage case and reinserts it into his or her mouth (element 108).

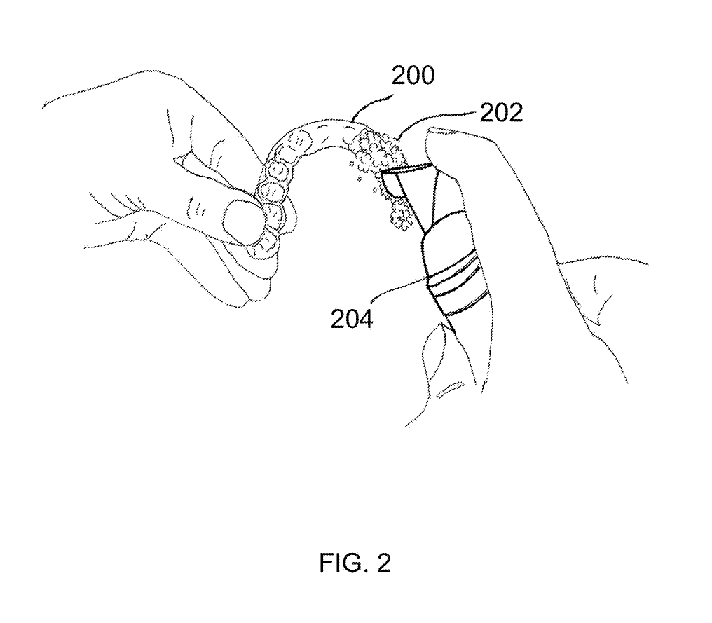

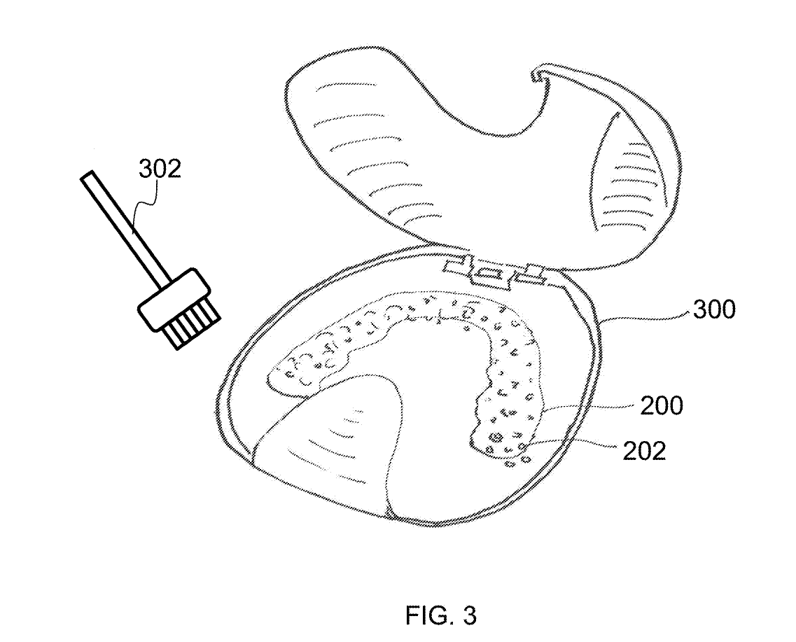

FIG. 2 illustrates the application of a lubricant formulation to an oral appliance 200 according to one embodiment of the invention. After removing the oral appliance 200—in this case, an orthodontic aligner—from his or her mouth, the user dispenses a quantity of lubricant formulation 202 as a foam from a foam-dispensing bottle 204. The lubricant formulation 202 is directed onto the oral appliance 200. Thereafter, the person reinserts the oral appliance 200 back into his or her mouth. Alternatively, the user stores the oral appliance 200 for a period of time in a storage case 300, as shown in FIG. 3. After a short period of time, the user retrieves the oral appliance 200 from the storage case 300 and reinserts it into his or her mouth. The lubricant formulation 202 can be spread onto the oral appliance 200 via a brush element 302. The brush element 302 can be used when the oral appliance 200 is either in the mouth or out of the mouth, such as in the storage case 300.

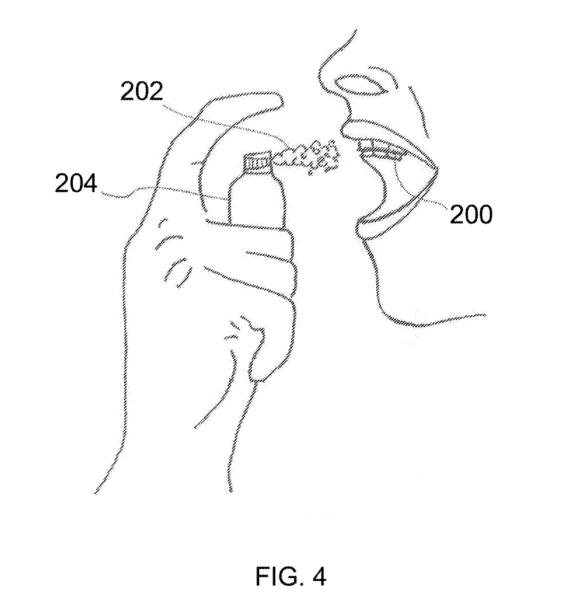

FIG. 4 depicts the appliance 200 wearer dispenses (e.g., sprays) a quantity of foamed lubricant formulation 202 from a foam-dispensing bottle 204, directly onto the oral appliance 200, in this case an orthodontic retainer. The user swishes the lubricant formulation 202 around in his or her mouth and then expectorates any remaining lubricant formulation 202.

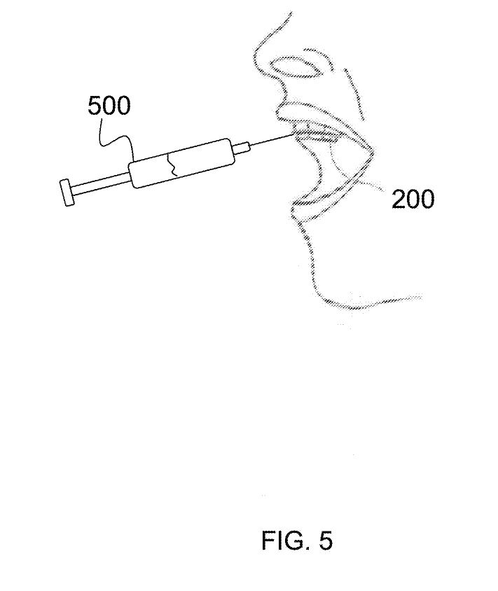

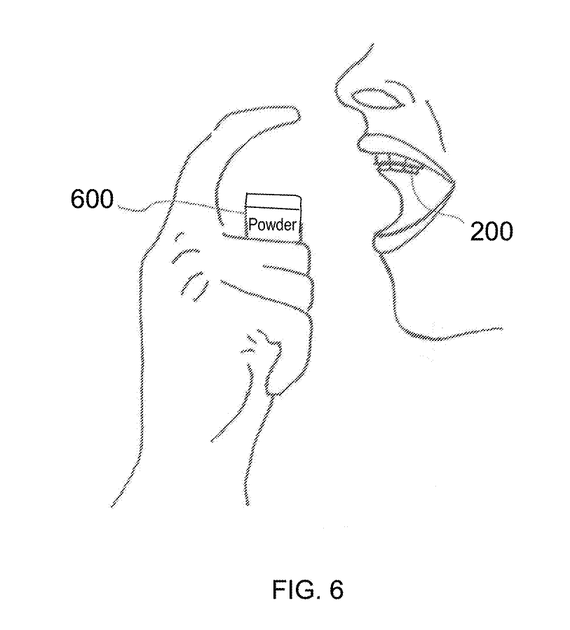

Alternatively, the lubricant formulation 202 can be applied via a syringe 500 to specific points either to the oral appliance 200 directly, to areas of the mouth/teeth that will contact the oral appliance 200, or both, inside the mouth or out of the mouth. This embodiment is depicted in FIG. 5. Finally, the lubricant formulation 202 can be in the form of a powder 600 (illustrated in FIG. 6) that the appliance wearer dispenses into the mouth which, when the powder 600 comes in contact with the wearer's saliva, dissolves in the mouth and disperses around the oral appliance 200.

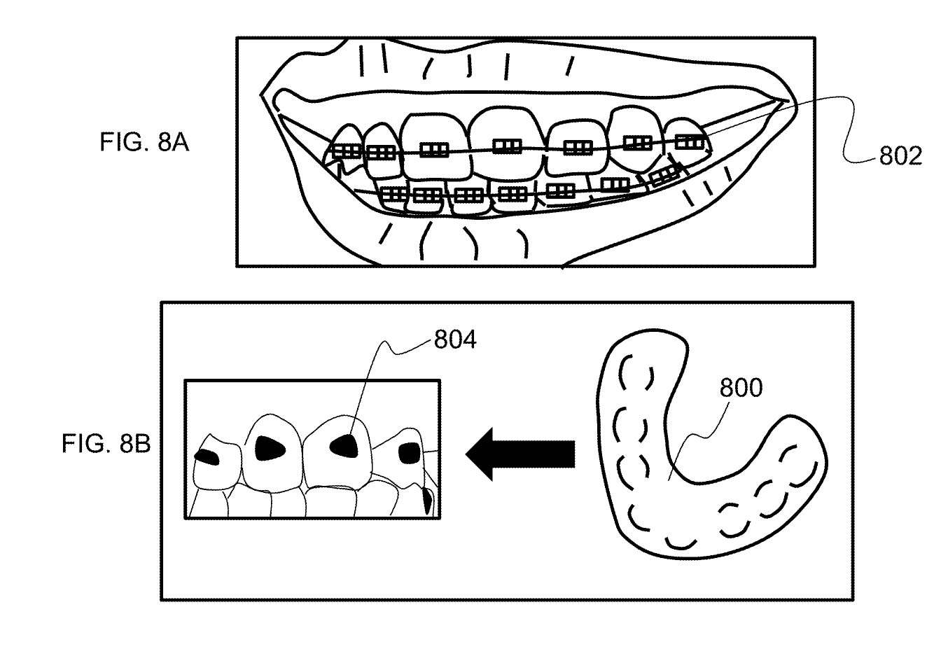

The lubricant formulation described herein reduces the friction between removable and fixed dental appliances. Dental appliances are designed to be installed on teeth, installed and removed, installed, removed, and replaced on teeth, or a combination of both. Regardless, when these appliances are activated, such as in the example when a dental tray is placed onto teeth having fixed appliances affixed, they move teeth. The dental appliances can be either removable or fixed on the teeth, or a combination of both fixed and removable. Examples of removable appliances include, but is not limited to, retainers, dental aligners (element 800 in FIG. 8B), dentures, sports guards, splints, flippers, stay plates, sleep apnea appliances, and orthodontic wires. Non-limiting examples of fixed appliances include orthodontic brackets (element 802 in FIG. 8A) and aligner attachments (element 804 in FIG. 8B).

In one embodiment, the lubricant formulation described herein interacts with the fixed appliance and the removable appliance together. As one example shown in FIG. 8B, an aligner attachment (element 804) is the fixed appliance, and the removable appliance is a plastic aligner (element 800), where a well within the plastic aligner (element 800) is where the fixed appliance and removable appliance meet (i.e., aligner attachment (element 804) interacts with aligner (element 800)) when the aligner (element 800) is placed in the mouth over the teeth and the aligner attachments (element 804). The lubricant formulation can be applied at this point of physical interaction or co-localization (e.g., well of aligner (element 800) or at the aligner attachment (element 804), or both) such that the lubricant interacts with the aligner (element 800) and aligner attachment (element 804) together to reduce friction in the mouth.

The lubricant formulation can work alone at lubricating the appliances to enhance the fit or movements, or, the lubricant formulation can be combined with other known energy sources, such as light radiation, vibration, pressure from chewing or swallowing (which results in the upper and lower teeth touching), pressure from chewing on a “chewie device”, and pressure from applying force to the appliances using digits or other objects. Energy from the body in the form of heat can change the properties of the formulation by decreasing viscosity, or activating ingredients making them more efficacious. Normal oral bacterial flora can be used as a catalyst to activate formulas.

The lubricant formulation to reduce friction can be applied to the oral appliances through one of the following techniques, some of which are described above. The formulation can be introduced into the mouth like a mouthwash and swished around the appliance(s); introduced into the mouth as a gel directly into the mouth and swished, wiped, or brushed; introduced into the mouth as powder that when comes in contact with saliva, dissolves in the mouth and disperses around appliances; introduced into the mouth on a brush, and applied to the appliances inside the mouth using a brush to spread the formulation; introduced into the mouth as a foam which is then swished around the appliances; introduced directly at or on the appliances, at points where they appliances interact, contact, or function with other parts of the appliances; introduced into one appliance and then carried to another appliance in the mouth; and introduced into a dental appliance with a syringe at specific friction points between the appliance and the tooth or orthodontic attachments affixed to teeth to facilitate the tooth movement. In another aspect, the lubricant formulation is applied at friction points between the appliance and an orthodontic fixed appliance. A fixed appliance is an orthodontic device that is affixed to the tooth or teeth which the patient does not remove, non-limiting examples of which include an orthodontic attachment, button, hook, or bracket. Multiple part A-B, A-B-C formulations can be used, mixed outside of the mouth using a double or triple barrel syringe mixing tip and then applied to appliances.

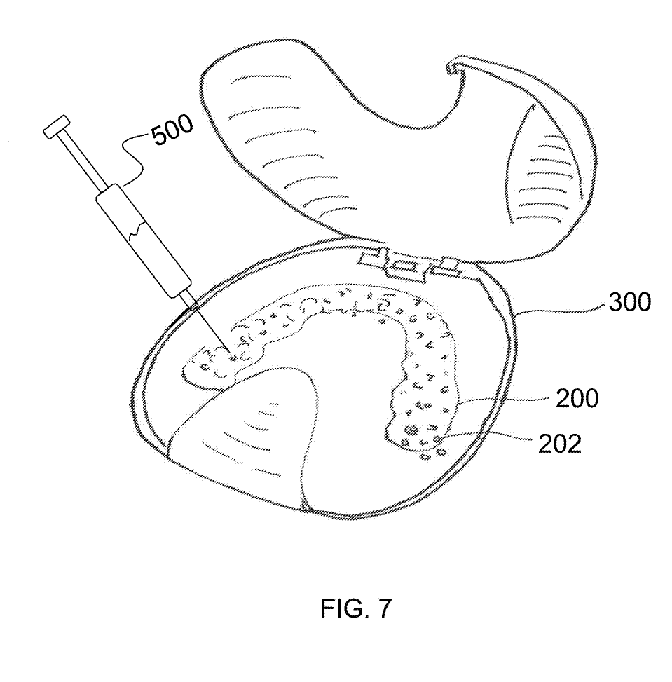

The formulation can be delivered to the appliance(s) through a syringe with various syringe tip sizes that are designed to fit into the appliance(s) and deliver the formulation at specific appliance sites where the formulation is needed, such as at points where the orthodontic braces connect to orthodontic wires, or where orthodontic aligners contact the attachments affixed to the teeth. As described above, the lubricant formulation can be placed in the mouth, swished, and then expectorated. The lubricant formulation can be placed in the mouth and then swished and then swallowed. The lubricant formulation can be placed in or onto the appliance and then swished and expectorated. Additionally, as depicted in FIG. 7, the lubricant formulation 202 can be placed in or onto the appliance 200 while outside the mouth via a syringe 500, swished, and then swallowed.

The lubricant formulation can be a stand-alone product or can be inserted into other types of products used in the mouth, including but not limited to mouth rinses, tooth pastes, breath mints, chewing gums, lozenges, troches, sprays, and gummies. The formulation itself can be produced in different forms, such as gels, pastes, liquids, powders, salves, creams, and gummies.

Additionally, the formulation can strictly be an appliance lubricant, or it contain other therapeutic properties, listed below. For instance, the formulation may contain one or more of the listed properties: anti-cavity; anti-tooth sensitivity; analgesic; tooth remineralization; anti-bacterial; anti-plaque; disinfecting; cleaning; anti-halitosis; and whitening. Moreover, the formulation can contain flavoring agents.

The formulation can contain lubricants used in other personal care products formulated for the skin, mouth, hair, or nails. The formulation can contain humectants used in other personal care products formulated for the skin, mouth, hair, or nails. The formulation can contain thickeners used in other personal care products formulated for the skin, mouth, hair, or nails. The formulation can contain natural or artificial flavors used in other personal care products formulated for the skin, mouth, hair, or nails. The formulation can contain emulsifiers used in other personal care products formulated for the skin, mouth, hair, or nails. The formulation can contain fluorides used in other personal care products formulated mouth. The formulation can contain antimicrobials used in other personal care products formulated for the skin, mouth, hair, or nails, including peroxides, chlorhexidine gluconate, antibiotics, antifungals, antivirals. The formulation can contain preservatives used in other personal care products formulated for the skin, mouth, hair, or nails. The formulation can contain anti-tooth sensitivity agents used in other personal care products formulated for mouth. The formulation can contain anti-calculus agents used in other personal care products formulated for mouth. The formulation can contain tooth whitening agents used in other personal care products formulated for mouth, including chemicals that are or break down into hydrogen peroxide, or other peroxides, releasing oxygen during their breakdown. The formulation can contain binding agents used in other personal care products formulated for the skin, mouth, hair, or nails. The formulation can contain enamel remineralization agents used in other personal care products formulated for mouth, such as amorphous calcium phosphate, Novamin, and milk derived agents. The formulation can contain emollient agents used in other personal care products formulated the skin, mouth, hair, or nails. The formulation can contain texture enhancers used in other personal care products formulated the skin, mouth, hair, or nails. The formulation can contain natural and artificial colors used in other personal care products formulated the skin, mouth, hair, or nails. The formulation can contain antioxidant agents used in other personal care products formulated the skin, mouth, hair, or nails. The formulation can contain plant extracts agents used in other personal care products formulated the skin, mouth, hair, or nails. The formulation can contain pigments used in other personal care products formulated the skin, mouth, hair, or nails. The formulation can contain emollient agents used in other personal care products formulated the skin, mouth, hair, or nails.

In one embodiment, the lubricant formulation also includes a cleanser formulation containing, on a percent-by-weight basis, 70 to 98% water, 0.01 to 5% surfactant(s), and 0.01 to 30% hydrogen peroxide, with the amount of each ingredient selected so that total ingredients sum to 100% by weight. In a more preferred embodiment, the cleanser formulation also contains one or more additional ingredient: 0.01 to 4% sweetener(s), 0.01 to 4% ethylenediaminetetraacetate (EDTA) or other metal binders, 0.01 to 6% sodium citrate or citric acid, and/or 0.01 to 3% flavorant(s), the amount of each ingredient being selected so that total ingredients sum to 100% by weight.

Hydrogen peroxide is an oxidizing germicidal agent and a bleaching agent, and plays a primary role in cleaning the oral appliance. The surfactant(s) facilitate the formation of a foam, as described below, and have intrinsic cleaning properties. One or a combination of surfactants is employed, including anionic, non-ionic, zwitterionic, poloxamer, and/or polysorbate surfactants. Nonlimiting examples include sodium lauryl sulfate (anionic), sodium dodecyl sulfate (anionic), cocamidopropyl betaine (zwitterionic), poloxamer 188, poloxamer 338, poloxamer 407, polysorbate 20, and alkyl polyglucosides (non-ionic).

Without being bound by theory, it is believed that EDTA facilitates the removal of inorganic debris (e.g., calcified deposits) through its chelating properties, and also functions as a preservative. Sodium citrate and citric acid are pH regulators and antioxidants. Preferred sweetener(s), if present, are those that do not promote tooth decay. Non-limiting examples include xylitol, aspartame, sucralose, sorbitol, and saccharine. Non-limiting examples of flavorants include peppermint, spearmint, wintergreen (methyl salicylate), cinnamon, vanilla, orange, and bubblegum.

Optionally, the lubricant and cleanser formulation includes one or more additional ingredients. Unless otherwise indicated, each ingredient is present in an amount of from 0.01 to 6.0% by weight, with the amount of each ingredient being selected so that total ingredients in the cleanser formulation sum to 100% by weight. Nonlimiting examples of such additional ingredients include other peroxides, e.g., carbamide peroxide, calcium peroxide, and magnesium peroxide; ethanol (0.001 to 50%); potassium hydroxide (0.001 to 10%); food coloring; sodium bicarbonate; fluorides, including acidulated, stannous, and sodium fluoride (0.0001 to 5%); remineralizing agents, e.g., calcium phosphate, amorphous calcium phosphate (ACP), and other calcium chemicals; cetyl pyridinium chloride and related quaternary ammonium chlorides (0.001 to 3%); triclosan (0.001 to 5%); desensitizing agents, e.g., potassium nitrate, potassium citrate, potassium chloride, stannous fluoride, and strontium chloride (0.001 to 11%); anti-halitosis agents, e.g., chlorine dioxide, essential oils, and zinc chloride; tartar control agents, e.g., pyrophosphate, hexametaphosphate, zinc, zinc chloride; sorbitol; propylene glycol (0.001 to 33%); disodium phosphate (0.001 to 29%); eucalyptol (0.001 to 3%); menthol (0.001 to 3%), and thymol (0.001 to 3%). In one embodiment, the lubricant and cleanser formulation contains only ingredients that are classified as Generally Recognized As Safe (GRAS) by the U.S. Food and Drug Administration.

The lubricant formulation is readily prepared by combining the ingredients in a suitable vessel, with stirring or other agitation sufficient to form a substantially homogeneous mixture. In a preferred embodiment, the cleanser formulation is provided in a conventional foam-dispensing bottle (also known as a foaming bottle, foam pump, foam-dispensing pump, etc.). Activating the pump causes the cleanser formulation to mix with air as it passes through the pump nozzle, fine pores or mesh, and/or other mechanism. In combination, the air and surfactant(s) cause the cleanser formulation to foam as it is ejected from the bottle. The hydrogen peroxide may also facilitate foaming.

The method of cleansing is suitable for use with all types of oral appliances, including orthodontic retainers, dental aligners (such as Invisalign® and ClearCorrect™ brand aligners), dentures, athletic mouth guards, snoring guards, sleep apnea appliances, tongue studs for pierced tongues, and other appliances commonly used in the mouth. In addition, the bleaching action of the hydrogen peroxide causes teeth, dental implants, dental crowns and bridges, and other dental prostheses to be whitened as the foamed cleanser formulation is applied, and the cleanser is further adapted to neutralize odors and kill pathogens that cause bad breath.

The formulation can be packaged in multiple sizes to accommodate users' needs, ranging from portability packaging (i.e., fit in a retainer or denture case, shirt or pants pocket, handbag or toiletry kit) to larger at home packaging sizes.

The viscosity of the formulation at the end of any dilution, dissolution, breakdown, from the form the product is sold prior to being applied to the appliance is between 1-200 centipoise, with the most ideal range of 1-50 centipoise. The formulation will be composed of water ranging from 10-99 percent.

Other modifications and variations may readily occur to those skilled in the art upon reading the present disclosure or seeing the invention in practice. It is intended that all such modifications and variations are included within the scope of the invention, which is limited only by the appended claims and equivalents thereof.

What is claimed is:

1. A method of lubricating an oral appliance while the appliance is in place in a user's mouth, comprising acts of:
introducing a quantity of a formulation into the user's mouth, the formulation being directed onto the appliance;
applying the formulation in such a way that the formulation will reside between the appliance and at least one of the user's teeth, wherein the formulation is aimed close to areas of contact between at least one of the user's teeth and the appliance; and
keeping the formulation in the mouth for a desired amount of time, wherein the formulation comprises a lubricant composition.

2. The method as set forth in claim 1, wherein the formulation is applied via a syringe between the appliance and at least one of the user's teeth.

3. The method as set forth in claim 1, wherein the formulation is applied via a syringe between the appliance and an orthodontic attachment affixed to at least one of the user's teeth.

4. The method as set forth in claim 1, wherein the formulation is applied via a spray mechanism.

5. The method of claim 1, wherein the formulation is applied via a foaming mechanism.

6. A method of lubricating an oral appliance while the appliance is in place in a user's mouth, comprising acts of:
introducing a quantity of a formulation into the user's mouth in such a way that the formulation will reside between the appliance and at least one of the user's teeth; and
keeping the formulation in the mouth for a desired amount of time, wherein the formulation comprises a lubricant composition.

7. The method as set forth in claim 6, further comprising acts of:
introducing the formulation into the user's mouth in powder form;
dissolving the powder in the user's mouth, resulting in a dissolved formulation; and
dispersing the dissolved formulation around the appliance.

8. The method as set forth in claim 6, further comprising an act of using a brush to spread the formulation onto the appliance.

9. The method as set forth in claim 6, wherein the formulation is applied via a foaming mechanism.

10. The method as set forth in claim 6, wherein the formulation is applied via a syringe.

11. A method of lubricating an oral appliance comprising acts of:
applying a quantity of a formulation in or onto the appliance while it is removed from a mouth in particular on portions of the appliance that come into contact with at least one of a user's teeth when the appliance is in the user's mouth;
inserting the appliance into the user's mouth; and
allowing the formulation to reside between the appliance and at least one of the user's teeth for a selected period of time, wherein the formulation comprises a lubricant composition.

12. The method as set forth in claim 11, wherein the formulation is applied via a spray mechanism.

13. The method of claim 11, wherein the formulation is applied via a foaming mechanism.

14. The method of claim 11, wherein the formulation is applied via a syringe.
